(19) United States
(12) Patent Application Publication
(10) Pub. No.: US 2008/0033412 A1
(43) Pub. Date: Feb. 7, 2008

(54) SYSTEM AND METHOD FOR CONVERGENT LIGHT THERAPY HAVING CONTROLLABLE DOSIMETRY

(76) Inventors: Harry Thomas Whelan, Whitefish Bay, WI (US); Perry B. Whelan, Whitefish Bay, WI (US); David P. Klemer, Whitefish Bay, WI (US)

Correspondence Address: QUARLES & BRADY LLP, 411 E. WISCONSIN AVENUE, SUITE 2040, MILWAUKEE, WI, 53202-4497, US

(21) Appl. No.: 11/829,142

(22) Filed: Jul. 27, 2007

Related U.S. Application Data
(60) Provisional application No. 60/820,980, filed on Aug. 1, 2006.

(51) Int. Cl. A61N 5/06; A61B 18/18

(52) U.S. Cl. 606/11; 607/90

(57) ABSTRACT

A system and method for providing a dose of irradiating light for a therapeutic process includes identifying an internal target area of a patient affected by a pathology and irradiating an externally accessible area of the patient proximate to the internal target area with a number of photons at least having wavelengths approximately within a near-infrared (IR) band. The method also includes receiving feedback from one of a spectrophotometer and a patient physiology monitoring system and adjusting the number of photons irradiating the externally accessible area of the patient. From the feedback, a determination is made to identify the number of photons needed to irradiate the externally accessible area of the patient to cause a change in biochemical state of cytochrome oxidase in the internal target area to a desired biochemical state of cytochrome oxidase in the internal target area.

### CROSS-REFERENCE TO RELATED APPLICATIONS

This application is based on provisional application 60/820,980 filed Aug. 1, 2006, entitled “SYSTEM AND METHOD FOR CONVERGENT LIGHT THERAPY HAVING CONTROLLABLE DOSIMETRY,” and claims the benefit thereof.

### STATEMENT REGARDING FEDERALLY SPONSORED RESEARCH

This invention was made with government support under Grant No. 5R21AT003002-02 from the National Center For Complementary and Alternative Medicine of the National Health Institute. The United States Government has certain rights in this invention.

### BACKGROUND OF THE INVENTION

The present invention relates generally to a system and method for providing therapeutic doses of light to a target area of a patient.

Neurodegenerative disorders, such as Parkinson's disease and Alzheimer's disease, are an increasing focus of treatment research. Current methodologies for addressing neurodegenerative disorders focus on using drugs or chronically implanted electrical stimulation devices to treat the symptoms of neurological disease. However, these devices are implanted late in the course of disease to treat specific symptoms. That is, these methodologies focus on treating specific symptoms of the disorders but do not seek to treat the root cause of the disorder. Accordingly, while the patient may receive temporary relief from the targeted symptoms, the disorder is permitted to progress.

Therefore, it would be desirable to have a system and method for therapeutically treating neurodegenerative disorders. That is, it would be desirable to therapeutically treat a neurodegenerative disorder as opposed to simply targeting specific symptoms that manifest as a result of the neurodegenerative disorder.

### SUMMARY OF THE INVENTION

The present invention overcomes the aforementioned drawbacks by providing a system and method for non-invasively providing a controllable, therapeutic dose of visible and/or near-infrared photons through deep tissue and into a specific target area within a patient. The system is designed to activate mitochondrial cytochrome c oxidase to induce regenerative activity within the target area and therapeutically treat degenerative disorders, including neurodegenerative disorders.

In accordance with one aspect of the invention, a method for determining a minimum effective dose of irradiating light for a therapeutic process is disclosed that includes identifying an internal target area of a patient affected by a pathology and irradiating an externally accessible area of the patient proximate to the internal target area with a number of photons at least having wavelengths approximately within a near-infrared (IR) band. The method also includes receiving feedback from one of a spectrophotometer and a patient physiology monitoring system and adjusting the number of photons irradiating the externally accessible area of the patient. Furthermore, the method includes determining, from the feedback, the number of photons irradiating the externally accessible area of the patient that begins to cause a change in biochemical state of cytochrome oxidase in the internal target area to a desired biochemical state of cytochrome oxidase in the internal target area.

In accordance with another aspect of the invention, a method of providing therapy for a neurodegenerative disorder is disclosed that includes identifying an internal target area of a patient associated with the neurodegenerative disorder and arranging a plurality of light sources about an externally accessible area of the patient proximate to an internal target area to direct photons emitted by the plurality of light sources in a converging pattern toward the internal target area of the patient. The method also includes irradiating the externally accessible are of the patient with a number of photons to deliver a dose of energy of between 2 mW/cm² and 10 mW/cm² to the internal target area.

In accordance with yet another aspect of the invention, a system for providing therapeutic doses of light to a target area of a patient is disclosed that includes a plurality of light sources configured to emit photons at least having wavelengths approximately within a near-IR band configured to direct the photons emitted by the plurality of light sources in toward an internal target area of a patient during a therapy session. Additionally, the system includes a control system configured to determine a change in biochemical state of cytochrome oxidase in the internal target area to a desired biochemical state of cytochrome oxidase in the internal target area.

In accordance with still another aspect of the invention, a system for providing therapeutic doses of light to a target area of a patient is disclosed that includes a plurality of light sources configured to emit photons at least having wavelengths approximately within a near-IR band configured to direct the photons emitted by the plurality of light sources in toward an internal target area of a patient during a therapy session. Furthermore, the system includes a control system configured to adjust a wavelength of the photons to cause a change in biochemical state of cytochrome oxidase in the internal target area to a desired biochemical state of cytochrome oxidase in the internal target area.

Various other features and advantages of the present invention will be made apparent from the following detailed description and the drawings.

### BRIEF DESCRIPTION OF THE DRAWINGS

FIG. 1 is a graph illustrating the spectral absorption properties of cytochrome c oxidase in the oxidized molecular state and reduced molecular state;

FIG. 2 is a perspective view of a convergent light therapy and control system in accordance with the present invention;

FIG. 3 is a schematic illustration of the convergent light therapy system and control systems of FIG. 2;

FIG. 4 is an illustration of an open-loop phototherapy configuration having two light sources;

FIG. 5 is a flow chart setting forth a method for performing a phototherapy session using the system of FIG. 4;

FIG. 6 is an illustration of a closed-loop phototherapy configuration including an active control system;

FIG. 7 is a flow chart setting forth a method for performing a phototherapy session using the system of FIG. 6;

FIG. 8 is an illustration of a closed-loop phototherapy system with adjunctive spectrophotometry including an active control system and real-time feedback system;

FIG. 9 is a flow chart setting forth a method for performing a phototherapy session using the system of FIG. 8; and

FIG. 10 is a flow chart setting forth a method for performing a phototherapy session using the systems of FIGS. 2-4, 6, and 8.

### DETAILED DESCRIPTION OF THE PREFERRED EMBODIMENT

The concept of an electromagnetic spectrum is an attempt to formalize and organize the notion of energy transmission across space and time, either via electromagnetic waves which serve to propagate energy, or equivalently, through transmission of particle-like photons. Wave-particle duality, a fundamental concept of quantum physics, ensures that these two views are, in fact, self-consistent and equivalent.

The wave/photon model of electromagnetic energy transmission incorporates the idea that energy is quantisized and is proportional to the frequency of the wave “packet” or photon. The energy carried by a single photon ‘particle’ in a vacuum is given by E=hv, where v is the frequency of the photon (or associated electromagnetic wave), and h is Planck's constant, given by 6.626×10-34 joule-seconds. Alternatively, the energy E can be written as E=hc/λ, where λ is the wavelength of the photon/wave and c is the velocity of light in a vacuum.

The portion of the electromagnetic spectrum for which photons possess wavelengths between 400 and 700 nanometers (nm) is of particular interest. Photons in this range possess sufficient energy to reach the posterior lining of the human eye, where they release their energy to rhodopsin molecules, triggering a cascade of biochemical reactions which ultimately results in the human perception of light. Indeed, the properties of photons in this so-called “visible region” of the electromagnetic spectrum and the biochemical reactions triggered by these photons are well-characterized and well-understood.

In contrast, there exists only a rudimentary understanding of analogous biochemical processes which are triggered in human cells by photons of ‘light’ radiation in the wavelength range 600-900 nm or “near-infrared” and lower “infrared” regions. One interaction which has come under intense scrutiny recently is the interaction of visible/near-infrared photons with mitochondrial proteins, such as cytochrome c oxidase. As will be described below with respect to the listed Examples, the energy exchange between incident photons and molecules of cytochrome c oxidase can affect significant, measurable, and reproducible changes at the cell, tissue, organ, and system level. Furthermore, these changes can, under certain circumstances, ameliorate pathological states of health, with beneficial results.

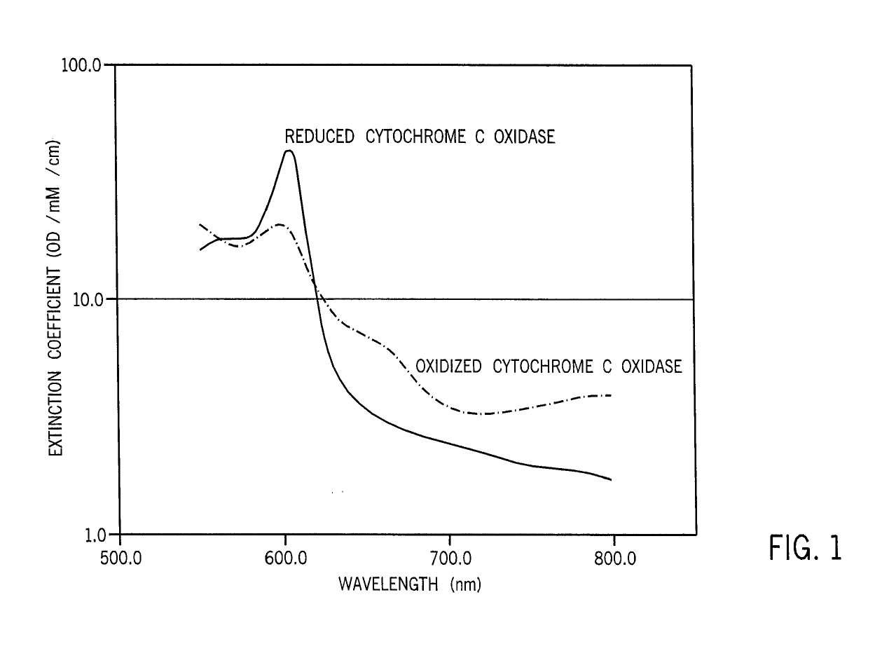

Referring to FIG. 1, the spectral absorption properties of cytochrome c oxidase in two molecular states, the oxidized and reduced forms, can be illustrated. As such, it is clear that the optical absorption properties of cytochrome c oxidase vary with both wavelength and molecular state. This is true except at discrete wavelengths where absorption is identical for both oxidation states, which is analogous to the so-called isobestic point of hemoglobin (another important physiologic protein). As will be discussed below, these optical absorption properties can be useful in extracting information on the biochemical state of cytochrome c oxidase, using non-invasive optical spectrophotometric measurements.

As will be described below with respect to the Examples, evidence has demonstrated that neurons exposed to light in the near-infrared spectrum can render such neurons more resistant to oxidative stress. Such types of stress are at the core of many neurodegenerative disorders such as Parkinson's disease and Alzheimer's disease. As will be described, the present invention provides a system and method for photobiomodulation by light in the red to near-infrared range (630-880 nm) and can improve recovery from ischemic injury in the heart, attenuate degeneration in the injured optic nerve, and protect against mitochondrial dysfunction in the retina.

As will be described, the present invention includes a near-infrared (IR) device designed to maximize internal tissue light dose (e.g., as applied to the brain), non-invasively, by convergence of multiple surface beams. Additionally, as will be described, the system is capable of delivering adequate doses of near-IR light evenly into the major internal organs of the body. In particular, the system is capable of delivering sufficient near-IR light deep into targeted tissues.

Mechanistic studies have shown that far-red to near-infrared light interacts with the enzyme cytochrome oxidase in mitochondria triggering signaling mechanisms which result in improved energy production, antioxidant protection, and cell survival. To this end, photobiomodulation will augment mitochondrial function and stimulate antioxidant protective pathways in cellular and animal models of Parkinson's Disease. Hence, as will be described below, the present invention is designed to therapeutically and non-invasively treat a targeted area of a patient to manifestly alter the course of a disease early on in the course of a development.

The present invention recognizes that nitric oxide is a molecule that can be used a signal of intercellular activity or change. For example, nitric oxide has a role in the control of blood flow and blood pressure via activation of the heme enzyme, soluble guanylate cyclase. Furthermore, the present invention recognizes that nitric oxide targets the mitochondrial oxygen-consuming heme/copper enzyme, cytochrome c oxidase.

As will be described, the present invention provides a system and method for identifying a minimal or minimum dose of near-IR light delivered to a target area to effectuate a desired biochemical state of cytochrome c oxidase using feedback from nitric oxide and dynamically controlling a dose of near-IR light delivered to the target area.

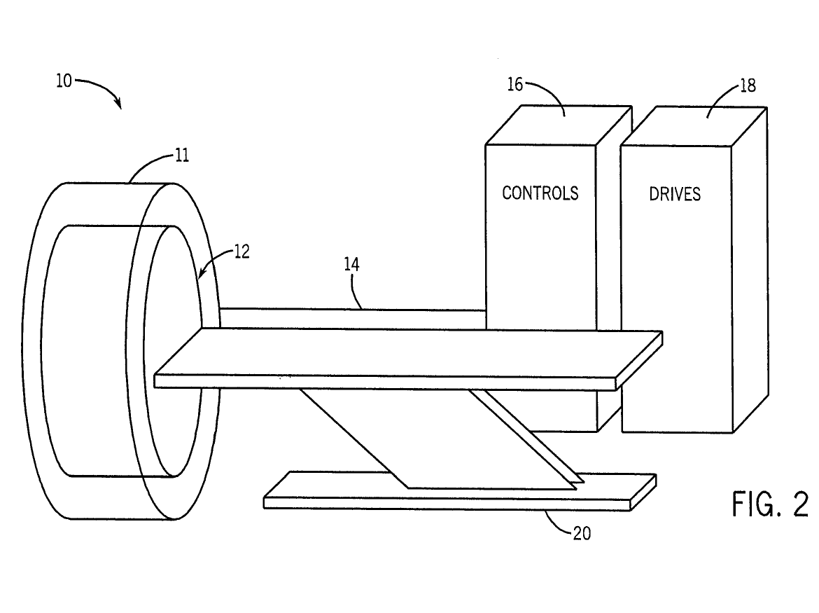

Referring now to FIG. 2, a convergent light therapy and control system 10 includes a gantry 11 that support an array (or multiple arrays) or light sources 12. It is contemplated that the light sources 12 may include laser light sources, super-luminescent diodes, light emitting diodes (LEDs), or the like. The components of the gantry 11 are driven and controlled through a communications and power connection 14 by a set of controls 16, 18 that will be described in detail below. Similarly, the controls 16, 18 may be connected to a patient table 20 through a communications and power connection 22 so that the position of the patient table 20 with respect to the gantry 11 can be dynamically controlled and adjusted.

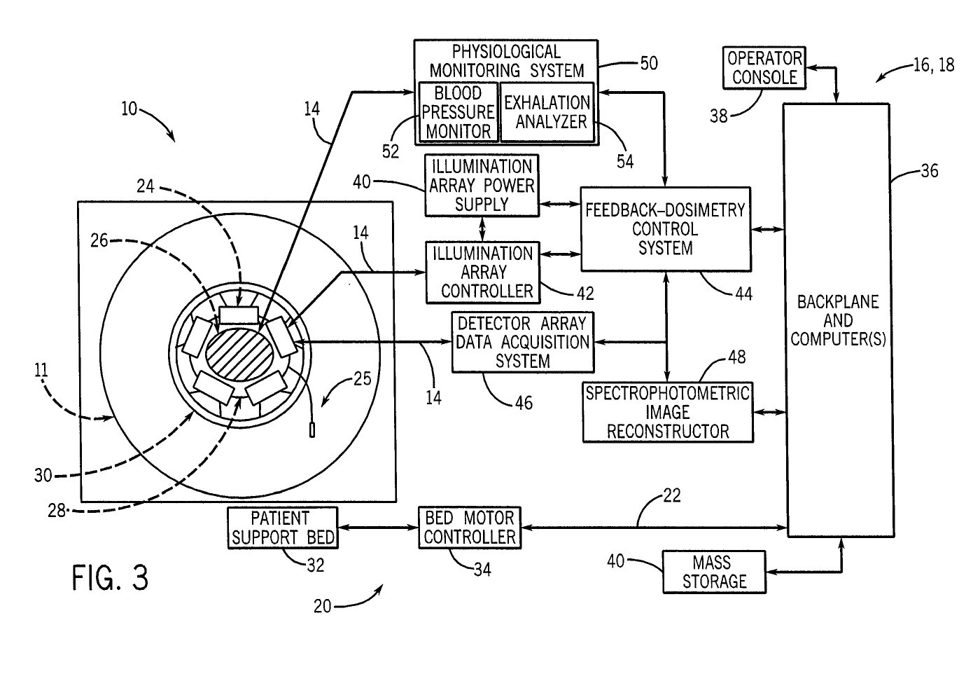

In particular, referring now to FIG. 3, the system 10 is shown in greater detail. For example, the gantry 11 includes an illumination/detector module with an integrated focusing/defocusing optic device 24, which is designed to direct light toward a patient 26 arranged within the gantry 11. Additionally, it is contemplated that a localized illumination module 25 may be included that is designed to be positioned within a cavity of the patient, for example, in the nasal cavity. The illumination/detector modules 24 are interconnected by way of a wiring harness 28 and supported by an electromechanical support framework 30.

To control the position of the patient 26, the patient table 20 is used that includes a patent support bed 32 that is dynamically controllable through a bed motor controller 34. In particular, as previously described, the components of the patient table 20 and the gantry 11 are controlled by way of connections 14, 22 to the control systems 16, 18.

The control systems 16, 18 include a variety of feedback, analysis, and control components that, as will be described, coordinate operation of the system 10 according to one of a variety of operational protocols. In particular, the control systems 16, 18 include a computer and/or backplane 36 that may include a computer, computer network, or other computing system/network. In this regard, it is contemplated that a dedicated operator console 38 may be included that provides a centralized station through which an operator can control a therapy session. The operator console 38 allows the operator to access and control a variety of components, such as a mass storage device 40 having stored therein resources including a priori data tables, or prescribe a therapy session. In the later case, the operator console 38 and backplane 36 coordinate operation of an illumination array power supply 40 and an illumination array controller 42. In accordance with some embodiments, the operation of the array power supply 40 and illumination array controller 42 are controlled by a feedback-dosimetry control system 44 that, in accordance with still other embodiments, coordinates operation of the system 10 using feedback from a detector array data acquisition system 46, spectrophotometric image reconstructor 48, and physiological monitoring system 50.

The physiological monitoring system 50 includes a blood-pressure monitor 52 and/or an exhalation analyzation system 54. Using blood-pressure monitoring 52, the physiological monitoring system 50 and/or feedback-dosimmetry control system 44 and/or the backplane and computers 36 identifies variation in blood-pressure indicative of the desired biochemical state of cytochrome oxidase in the internal target area of the patient that are caused by the proper dose and wavelength of near-IR light transferring the proper amount of energy to the target area. For example, the system seeks to identify a drop in blood pressure indicative of the activation of the heme enzyme, soluble guanylate cyclase. Additionally or alternatively, exhalation analyzation system 54 can be used to identify the presence of nitric oxide in the patient's breath that is indicative of the proper dose and wavelength of near-IR light transferring the proper amount of energy to the target area and causing nitric oxide disassociate with cytochrome c oxidase. As will be described, this feedback can be used to non-invasively identify the dose of near-IR light entering the target area and, furthermore, identify a minimum dose of near-IR light needed to perform the desired therapy on the target area.

It is contemplated that the above-described system 10 and treatment process could be implemented in a variety of configurations that may or may not utilize all of the components described above. For example, the instrumentation for generating the visible and near-infrared light to activate cytochrome c oxidase could be arranged as an open-loop visible/near-IR phototherapy device that determines the proper delivery dose based on a priori tissue estimates of scattering and absorption properties. Alternatively, it is contemplated that the instrumentation for generating the visible and near-infrared light may be arranged as a closed-loop visible/near-IR phototherapy device that determines the proper delivery dose based on real-time in-vivo measurements of transmitted and reflected components of optical signals. Also, it is contemplated that the closed-loop phototherapy device may determine the proper delivery dose based on feedback dosimetry as well as an adjunctive spectrophotometric determination of cytochrome c oxidase oxidation states.

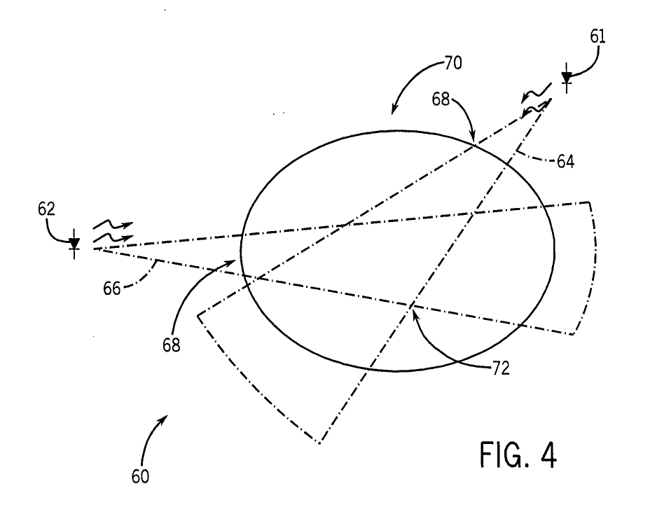

Referring now to FIG. 4, an open-loop visible/near-IR phototherapy implementation 60 is shown, for simplicity, as having only two light sources 61, 62 configured to emit respective beams of light 64, 66 that are directed toward an externally accessible area 68 of a patient 70 to converge toward an internal target area 72 in the patient 70. It should be noted that while for simplicity only two light sources 61, 62 are shown, an actual implementation would include many additional light sources arranged to have a convergent beam pattern.

Within this open-loop configuration 60, the photons transmitted or reflected during treatment are not measured. In this regard, the open-loop system 60 is highly cost effective and robust. To control the dose light delivered by the beams 64, 66, the system 60 relies on a priori knowledge of tissue optical properties, as related by tissue absorption and scattering coefficients. Fortunately, given the relatively benign effects of visible (e.g., red) and near-IR radiation, over-treatment of tissue may result in little or no adverse effects at the photon intensity levels utilized (i.e., the therapeutic index of visible/near-IR phototherapy is quite high at the power levels of interest). Accordingly, the predominant concern in the case of the open-loop system 60 is that the incident energy exceeds a minimal level capable of resulting in a therapeutic benefit at a local tissue region of interest.

Proper performance and therapeutic response of the system 60 depends significantly on accurate knowledge of the local optical properties of the human tissue being treated. Table 1 provides example data for absorption and scattering coefficients relevant to phototherapy applied in a neurological application.

| | | absorption | scattering |
|---|---|---|---|
| | | coefficient | coefficient |
| | wavelength | (/mm) | (/mm) |
| cortex | 811 | 0.0182 | 0.74 |
| (frontal) | 849 | 0.0185 | 0.74 |
| | 956 | 0.0206 | 0.8 |
| brain | 674 | 0.0179 | 0.99 |
| cortex | 811 | 0.019 | 0.48 |
| (temporal) | 849 | 0.0179 | 0.45 |
| | 956 | 0.0218 | 0.42 |
| brain | 674 | 0.0165 | 1.34 |
| white matter | 849 | 0.0132 | 0.98 |
| (cerebellar) | 956 | 0.0299 | 0.84 |

The administration of a desired photon energy density at a given location of tissue may be estimated from these values, as a function of wavelength. Assuming a simple one-dimensional approximation, one may estimate the photon intensity as a function of depth using a standard exponential model:

I(z)=Iₒe^{-(α+α}^{ₛ}^{)z} Eqn. 1;

Where α and αₛ are respectively the absorption and scattering coefficients of the local tissue, z is the depth in the tissue, and Iₒ is the intensity incident upon the tissue. The penetration depth at which I(z) decreases to 1/e of the incident value is a function of wavelength, which ranges from approximately 5 mm at 1064 nm to approximately 1 mm at 488 nm.

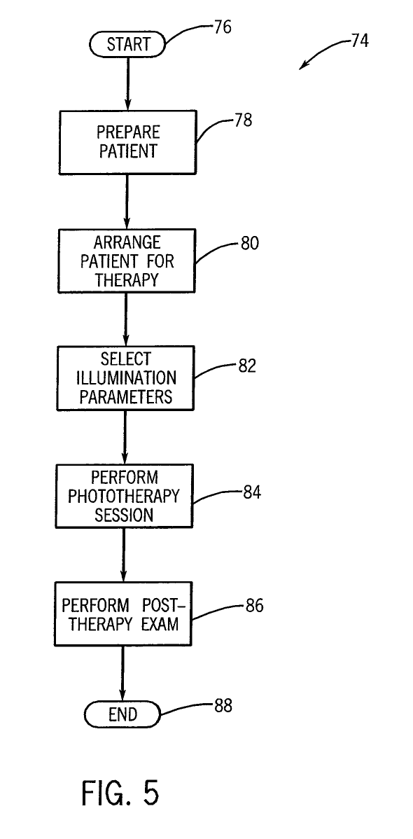

Referring again to FIGS. 3-5, the specific steps performed to carry out a therapy session using the open-loop configuration 60 are set forth in the flow chart of FIG. 5. The process 74 begins 76 by preparing the patient 78 for the therapy session. The preparation 78 may include an explanation of the principles of operation, along with a discussion of the risks and benefits of the procedure, in the usual fashion for any medical procedure. Thereafter, the patient is asked to lie recumbent on the patient support bed and the phototherapy system is configured for therapy. For example the bed and patient may be advanced into the phototherapy gantry and/or a local phototherapy probe may be arranged. It is contemplated that the local phototherapy probe may be designed to access an externally accessible cavity of the patient, such as the nasal passage, to position a light source as close as possible to the desired target area without the need to surgically position the probe. Additionally or alternatively, the local probe may be formed as a “bonnet” or “shower-cap” array that is positioned on the head or about another portion of the patient.

Once these setup procedures are complete, a main power delivery system is activated so as to apply power to the illumination array power supply, operator console, main computer/backplane, and all subcomponents of the phototherapy system are active and the operator enters appropriate parameters for illumination 82. As illustrated in FIG. 3, this may be completed using the operator console, which (via the computer/backplane 36) applies the entered settings to the illumination array power supply 40 and illumination array controller 42. According to use in the open-loop configuration 60, the feedback dosimetry control system/computer 44 is not utilized, and is effectively bypassed. Similarly, according to use in the open-loop configuration 60, the spectrophotometric image reconstructor 48 and physiological monitoring system 50 are not utilized and are effectively bypassed. To this end, the operator simply enters a desired wavelength, duration, and intensity to select the therapy parameters 82 when using the system in the open-loop mode. As will be described, it is contemplated that one desired set of operational parameters would include selecting an illumination intensity at the externally accessible area proximate to the internal target area designed to deliver approximately 5 mW/cm² to the internal target area for a duration of approximately 3 minutes using photons having a wavelength of 670 nm.

After the settings are applied 82, the modules are activated and the illumination is applied to the patient's anatomy (e.g., cranium and brain) for the given length of time 84. Again, the illumination sources in the illumination/detection modules may be LED sources, superluminescent diodes (SLDs), solid state laser diodes, or other light sources. The detector elements (e.g. photodiodes, phototransistors, photoresistors, etc.) in the illumination/detection modules are used to record transmitted and scattered light. This information is passed to the detector array data acquisition system 46 of FIG. 3. However, according to use in the open-loop configuration, this information is merely recorded as information, and is not used in the control of system function. The computer/backplane 36 saves all relevant control parameters and data acquired by the detector array data acquisition system onto mass storage 40. After completion of the therapy session 84, the illumination/detection modules are deactivated, the patient bed is retracted from the gantry, and the patient is informed that therapy has concluded. Following the therapy session 84, the results of the phototherapy are evaluated 86 through an examination procedure and the process concludes 88.

Referring now to FIG. 5, it is contemplated that the open-loop system 60 of FIG. 4 may be augmented to form a closed-loop system 90 including a plurality of phototransistors 92, 94 designed to monitor the power transmitted by the beams 64, 66. Using the photosensors 92, 94, transmitted and scattered (reflected) light can be measured. It is contemplated that the photosensors 92, 94 may be photodiode, phototransistor, avalanche photodiode, photomultiplier tube, CCD (charge-coupled device) camera, or other such devices. In any case, the photosensors 92, 94 are connected to feedback/control lines 96 to provide feedback that can be used to perform active control (e.g., amplitude control, etc.) of the dose (e.g., intensity, duration, wavelength, etc.) delivered by the light sources 61, 62. That is, the measured signals are processed in real time and used to directly modulate the intensity of the illuminating sources. This technique is less dependent on a priori knowledge of tissue optical properties and can readily accommodate dynamic changes in scattering and absorption properties of tissue, which, for example, may occur with changes in local blood (hemoglobin) volume and hemoglobin oxygenation states.

Additionally or alternatively, a patient physiology monitor 98 may be used as a feedback source that is connected to feedback/control lines 96 to provide feedback that can be used to perform active control (e.g., amplitude control, etc.) of the dose (e.g., intensity, duration, wavelength, etc.) delivered by the light sources 61, 62. The patient physiology monitor 98 may monitor one or more aspects of the patent for signs of the effective dose and amount of energy delivered to the internal target area 72. For example, the patient physiology monitor 98 may analyze air exhaled by the patient 70 to identify an increased concentration of nitric oxide in the exhaled air because nitric oxide is a molecule that can be used a signal of intercellular activity or change. To this end, when an increase in nitric oxide is detected, it indicates that at least a minimum number of photons in the beams 64, 66 are reaching the internal target area 72. Additionally, it is contemplated that the patient physiology monitor 98 may include a blood pressure monitor because nitric oxide has a role in the control of blood flow and blood pressure via activation of the heme enzyme, soluble guanylate cyclase. Accordingly, the present invention recognizes that nitric oxide targets the mitochondrial oxygen-consuming heme/copper enzyme, cytochrome c oxidase.

In the closed-loop phototherapy system 90, a priori knowledge of absorption and scattering coefficients is not necessary. Instead, the photosensors 92, 94 and/or the patient physiology monitor 98 are used to measure or determine the transmitted or scattered radiation. With respect to using the photosensors 92, 94 for active control, using Eqn. 1, two measurements of intensity can be made, at z=0 (the entry point of the incident radiation) and z=D (the point at which the radiation leaves the tissue after passing through a distance D), as follows:

I(0)=Iₒ Eqn. 2;

I(D)=Iₒe^{-(α+α}^{ₛ}^{)D} Eqn. 3.

Accordingly, the total optical loss (α+αₛ) can be estimated as:

(α+αₛ)=D⁻¹·ln(I(0)/I(D)) Eqn. 4.

This value of total optical loss may, in turn, be used to estimate energy density as a function of tissue depth. Thus, the system can affect real-time dosimetry through modulation of the administering optical sources.

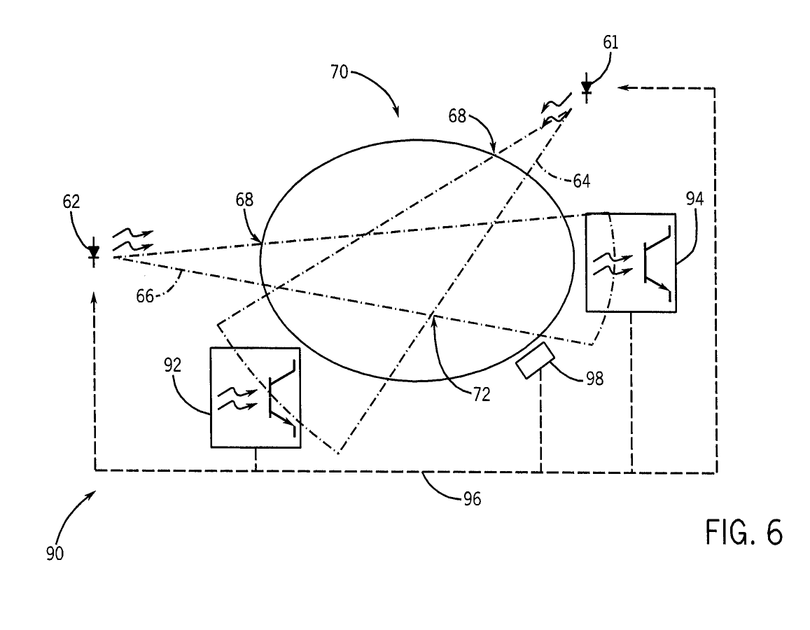

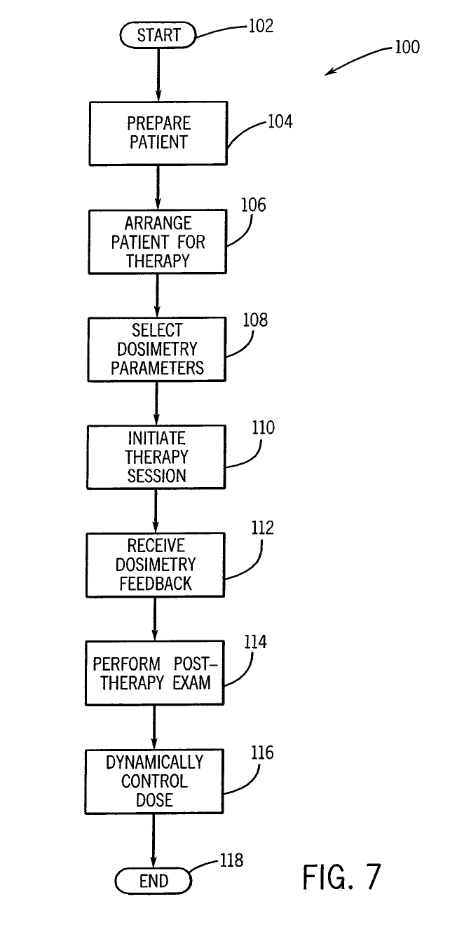

Referring to FIG. 7, the process 100 performed to carry out a therapy session using the closed-loop system of FIG. 6 begins 102 by preparing the patient 104 for the therapy session. The preparation 104 may include an explanation of the principles of operation, along with a discussion of the risks and benefits of the procedure, in the usual fashion for any medical procedure. Thereafter, the patient is asked to lie recumbent on the patient support bed and the phototherapy system is configured for therapy. For example the bed and patient may be advanced into the phototherapy gantry and/or a local phototherapy probe may be arranged. It is contemplated that the local phototherapy probe may be designed to access an externally accessible cavity of the patient, such as the nasal passage, to position a light source as close as possible to the desired target area without the need to surgically position the probe. Additionally or alternatively, the local probe may be formed as a “bonnet” or “shower-cap” array that is positioned on the head or about another portion of the patient.

Once these setup procedures are complete, a main power delivery system is activated so as to apply power to the illumination array power supply, operator console, main computer/backplane, and all subcomponents of the phototherapy system are active and the operator enters appropriate parameters for dosimetry 108. That is, unlike the open-loop operational procedure described with respect to FIG. 5 where the operator entered parameters for illumination, the closed-loop operational procedure 100 includes entering the parameters for dosimetry 108. As illustrated in FIG. 3, this may be completed using the operator console, which (via the computer/backplane 36) applies the entered settings to the illumination array power supply 40 and illumination array controller 42. According to use in the open-loop configuration 60, the feedback dosimetry control system/computer 44 is not utilized, and is effectively bypassed. Similarly to the open-loop configuration 60, the spectrophotometric image reconstructor 48 and physiological monitoring system 50 are not utilized and are effectively bypassed. To this end, the operator enters a desired wavelength, duration, and dose to select the therapy parameters 82. As will be described, it is contemplated that one desired set of operational parameters 108 would include selecting (or identifying) a dose of 5 mW/cm² delivered to the internal target area for a duration of approximately 3 minutes using photons having a wavelength of 670 nm.

After the settings are applied 108, the modules are activated and the illumination is applied to the patient's anatomy (e.g., cranium and brain) 110. To achieve the desired dose, the detector elements (e.g. photodiodes, phototransistors, photoresistors, etc.) in the illumination/detection modules are used to record transmitted and scattered light and/or feedback from the physiological monitoring systems are processed 112 and the illumination intensity delivered to the patient is dynamically controlled 114. As will be described, the feedback 112 and dynamic control of dose 114 based on the feedback may not only be utilized to deliver the desired dose to the internal target area, but may be used to identify a minimum effective dose for an individual patient.

After completion of the therapy session 112-114, the illumination/detection modules are deactivated and the results of the phototherapy are evaluated 116 through an examination procedure and the process concludes 118.

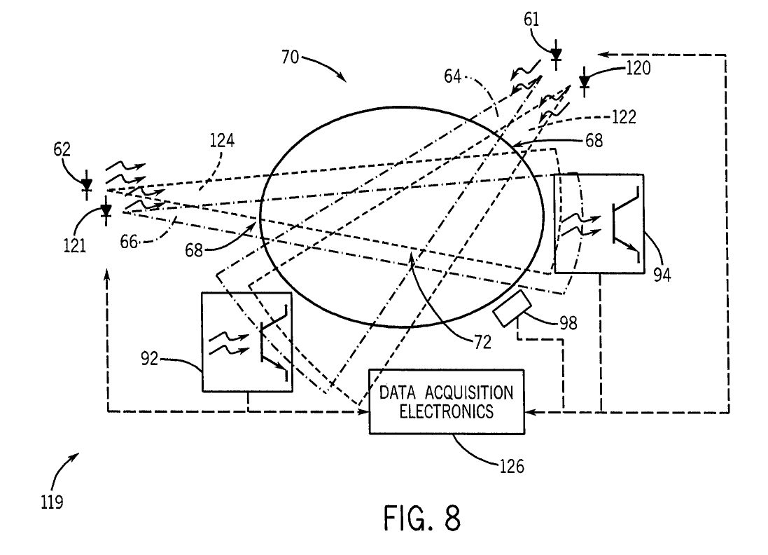

Referring now to FIG. 8, the closed-loop phototherapy system 90 of FIG. 6 may be coupled with feedback dosimetry and adjunctive spectrophotometry to create a system 119 capable of utilizing and controlling multiple sources 61, 62, 120, 121 designed to emit beams 64, 66, 122, 124 having various wavelengths. Specifically, it is contemplated that one pair of sources 61, 62 generates beams 64, 66 with a first wavelength and another pair of sources 120, 121 generates beams 122, 124 of a second wavelength different from the first wavelength. These monochromatic sources 61, 62, 120, 121 may include solid-state laser diodes, superluminescent diodes, or LEDs and are designed to combine optical energies.

As described with respect to FIG. 1, optical absorption is dependent on wavelength and the oxidation state of cytochrome c oxidase. As a result, optical measurements at more than one wavelength may be used to calculate individual contributions of optical absorption from each oxidation state of cytochrome c oxidase.

In simplified terms, the system 119 includes a system for phototherapy administration as well as an imaging modality, in which voxel-by-voxel determination of oxidation states of cytochrome c oxidase are reconstructed using techniques similar to those employed in diffuse optical tomography. In this regard, the system 119 may be considered a photo-tomotherapy system.

The system 119 may be extended to more complex systems comprised of multiple optical absorbers/scatterers, such as cytochrome c oxidase, hemoglobin (in its oxygenated and deoxygenated states) and water, by extending the number of source wavelengths. However, the number of discrete wavelengths chosen depends on a number of considerations. For example, one consideration includes the number of biochemical species to be determined from optical measurements (e.g., oxidized and reduced cytochrome c oxidase, oxy- and deoxyhemoglobin, water). Additionally, the number of excess or redundant data points utilized to improve the accuracy of the measurements, through inverse solution of an over-determined set of data, should also be considered. Furthermore, the particular techniques used to extract individual wavelength data and the limitations of any associated electronics 126, including circuit speed limitations in time-division multiplexing schemes or bandwidth limitations in frequency-division multiplexing schemes, should also be considered. To these ends and others, the overall system complexity and cost constraints will aid the determination of the number of discrete wavelengths utilized by the system 116.

Using multiple wavelengths to perform spectrophotometric measurements in addition to the dosimetry techniques described above adds an additional and powerful tool to the phototherapy system 116. In particular, the system 116 is capable of noninvasive measurement of the underlying biochemical processes. The techniques for this determination rely on an inverse solution of a three-dimensional partial differential diffusion equation describing photon migration through an absorbing/scattering medium, which is given by:

∇·D(r)∇Φ(r,ω)−[μₐ(r)−iω/c] (r,ω)=−S(r,ω) Eqn. 5;

where Φ is the photon density as a function of frequency (wavelength) and distance into the medium, D is the diffusion coefficient (a function of absorption and scattering coefficients), and S represents the source distribution. Therefore, by applying a known distribution of source radiation and measuring the photon flux as it exits the tissue volume under consideration, it is possible to determine D (i.e., absorption and scattering properties) through an inverse solution of the differential equation above.

While serving as a powerful spectrophotometric technique, this approach is computationally intensive, requiring high-speed computational equipment for real-time implementation. However, a number of solutions have been presented to address efficient approaches to the solution of the above differential equation, as well as questions of the existence and uniqueness of the inverse solutions thereof.

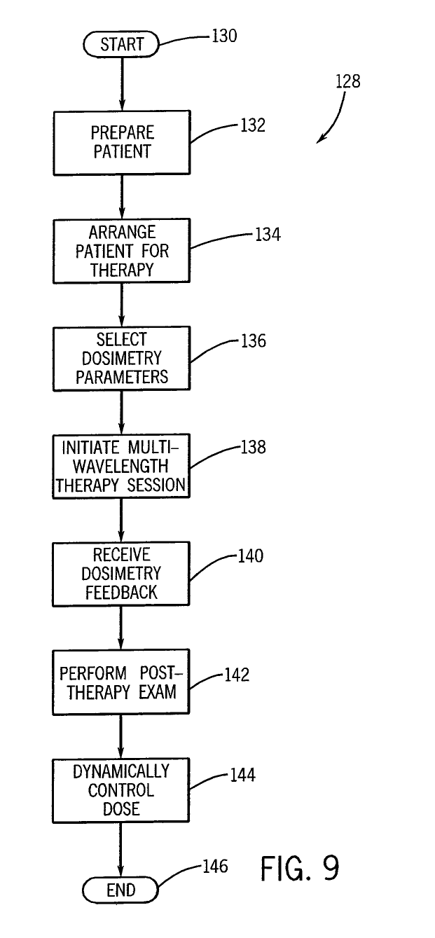

Referring now to FIG. 9, the specific steps 128 performed to carry out a therapy session using the closed-loop system 119 of FIG. 8 start 130 with preparing the patient 132 and arranging the patient for therapy 134, as described above. Thereafter, the operator enters appropriate parameters for phototherapy dosimetry parameters 136 in a manner similar to that described above with respect to FIG. 7. However, using the closed-loop system 119 of FIG. 8, the operator may select one, two, or more wavelengths. After the dosimetry parameters are selected 136, illumination may be applied from the sources to provide a multi-wavelength therapy 138.

In accordance with one embodiment, the multi-wavelength therapy session 138 includes a time-division-multiplexed delivery method, in which all sources at ‘wavelength 1’ are first applied for a given interval, then all sources at ‘wavelength 2’ for a given interval, then all sources at ‘wavelength 3’ for a given interval, and so on, in a repetitive cycle. Alternatively, the multi-wavelength therapy session 138 may include frequency-division-multiplexed delivery method, in which sources of ‘wavelength 1,’ ‘wavelength 2,’ ‘wavelength 3,’ and so on are encoded with a uniquely-identifying modulation frequency that is significantly lower than the frequency of the unmodulated light signal. Also, it is contemplated that other delivery methods may be utilized, for example, in combined time and frequency modulation.

During the multi-wavelength therapy session 138, dosimetry feedback is received from the physiology monitoring system and the detector elements (e.g. photodiodes, phototransistors, photoresistors, etc.) in the illumination/detection modules. The feedback from the detector elements is used to record transmitted and scattered light for each of the individual source wavelengths. This is accomplished using time-division-multiplexed signal receivers, frequency-division-multiplexed signal receivers, or other schemes to determine transmitted and scattered light for each individual source wavelength. This information is passed to the detector array data acquisition system. In accordance with this configuration and mode of operation, the information recorded by the detector array data acquisition system 46 is used to adjust parameters for the illumination array power supply and illumination array controller in real-time during the therapy session, so as to achieve a desired dose within the internal target tissue 142. The dosimetry feedback in this arrangement and operation may utilize information about scattered/transmitted light at one, two, or more of the source wavelengths. In particular, the spectrophotometric image reconstructor 48 of FIG. 3 is active and uses the detected signal information acquired by the detector array data acquisition system 46, at one, two, or more of the source wavelengths, in order to compute relative percentages of cytochrome c oxidase concentration.

After completion of the therapy session 138-142, the illumination/detection modules are deactivated and the results of the phototherapy are evaluated 144 through an examination procedure and the process concludes 146.

Therefore, the above-described systems and methods provide a variety of designs and implementations for the administration of phototherapy, along with “imaging-feedback” techniques for dosimetry and spectrophotometry. While the open-loop system is cost-effective and computationally simple, it depends on accurate a priori knowledge of tissue properties. The closed-loop systems provide accurate dosing. When coupled with spectrophotometric techniques, the systems are capable of direct, non-invasive monitoring of the underlying biochemical processes.

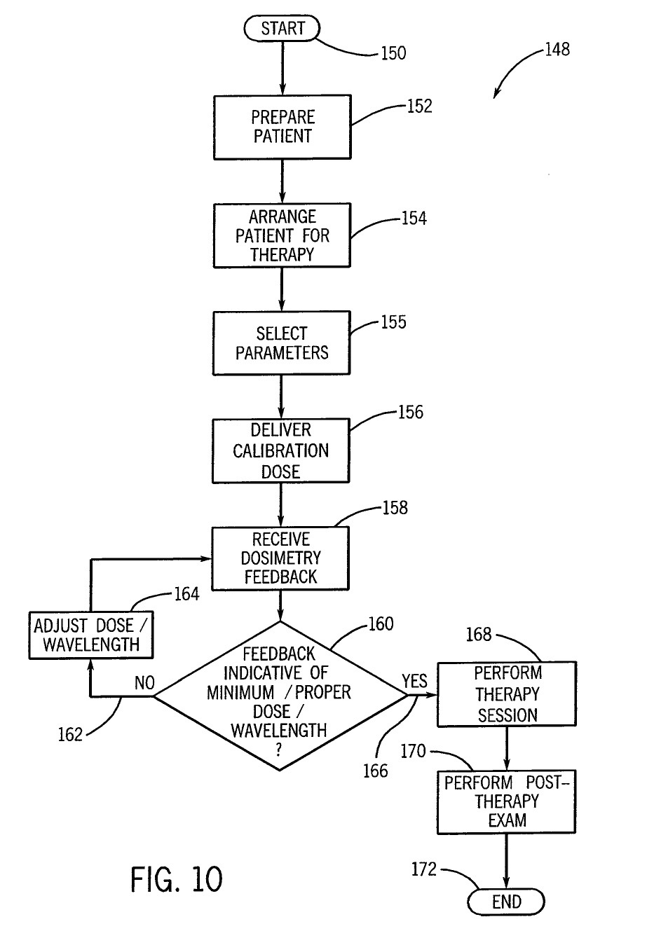

The feedback and control systems described above may not only be used to maintain a proper dose during a therapy session, it is contemplated that the feedback and control systems may be utilized to determine a desired and/or minimum effective dose. Referring now to FIG. 10, a process 148 for identifying and providing a minimum effective dose at an internal target area of a patient begins 150 by preparing the patient for therapy 152. Again, this includes informing the patient about the procedure, identifying a target area, and the like. Thereafter, the patient is arranged for therapy 154, which includes arranging the light sources and feedback devices and, if known, parameters for the therapy may be entered 155. As will be described, it is contemplated that the above-described feedback systems may be used to determine the desired parameters. Once the patient and system positioned for therapy 154 and any known parameters entered 155, a calibration dose of light may be provided to the internal target area 156 and feedback about the dose delivered to the internal target area is monitored 158.

Specifically, as described above, feedback is received from the physiological monitoring system 50 of FIG. 3, including feedback from the blood-pressure monitor 52 and/or the exhalation analyzation system 54. Using the blood-pressure monitor 52, the physiological monitoring system 50 and/or feedback-dosimmetry control system 44 and/or the backplane and computers 36 looks for variation in blood-pressure indicative of the desired biochemical state of cytochrome oxidase in the internal target area of the patient that are caused by the proper dose and wavelength of near-IR light transferring the proper amount of energy to the target area 160. For example, the system seeks to identify a drop in blood pressure indicative of the activation of the heme enzyme, soluble guanylate cyclase. Additionally or alternatively, exhalation analyzation system 54 can be used to identify the presence of nitric oxide in the patient's breath that is indicative of the proper dose and wavelength of near-IR light transferring the proper amount of energy to the target area and causing nitric oxide disassociate with cytochrome c oxidase.

By providing a very low initial/calibration dose 156, it can be expected that the initial feedback will not indicate a proper dose level 162 and the dose is slightly increased 164. By repeatedly increasing the dose 164 and monitoring the dose feedback 158, the system can determine when the proper/minimum dose is delivered 160, 166. This same process may be used to identify other parameters for a therapy session, such as wavelength, and the like. For example, instead of starting at a minimum dose and incrementally increasing the dose, a base wavelength may be initially used and incrementally adjusted to identify an optimum wavelength. To this end, the feedback systems can be used to non-invasively identify desirable operational parameters of near-IR light entering the target area, such as identifying a minimum dose of near-IR light needed to perform the desired therapy on the target area.

Once the desired operational parameters are selected/determined, the therapy session is performed 168, for example, for a desired duration of approximately 3 minutes. That is, in developing the present invention, it was determined that extended durations of therapy sessions, for example, extending beyond 10 to 30 minutes and, in some cases, extending beyond only 4 to 5 minutes provided no additional benefit beyond that initially gained by the first 3 to 4 minutes. Following the therapy session 168, the patient is evaluated 170 and the process ends 172. However, it is contemplated that the above-described therapy process 148 is part of an overall therapy regiment. That is, the development of the present invention also identified that, while extended therapy sessions showed diminishing returns, multiple individual therapy session at regular intervals is quite beneficial. Therefore, it is contemplated that the above-described processes may be performed multiple times per day, for example twice a day, every day.

### EXAMPLES

The above-described systems are capable of producing near-IR light at a wavelength, for example, 670 nm, to provide a treatment that attenuates cytotoxity and dopaminergic cell death in a patient with Parkinson's disease and significantly improves clinical outcome. The specific wavelength of the near-IR light is selected based on the particular pathology for which the treatment is targeted and may be determined from a priori knowledge or may be determined using the above-described feedback systems. As will be shown below, in the case of Parkinson's Disease, which combines genetic susceptibility and mitochondrial toxicity, a wavelength of approximately 670 nm has been determined to be desirable.

To determine a desirable wavelength, confluent cultures of human dopaminergic cells (SH-SY5Y) engineered to stabily overexpress the A30P mutant form of α-synuclein were exposed to increasing concentrations of the dopaminergic toxin MPP+ (0-5 mM) for 14 hours. Cell proliferation (protein concentration and MTS assay), mitochondrial function (mitochondrial dehydrogenase activity), oxidative stress (H2O2 production), and cell viability (LDH release) were assessed 12 hours later. Experimental cultures were treated with 670 nm LED arrays (GaAlAs LED; bandwidth 670+25 nm at 50% power). An LED array can be created from LEDs that are commercially available, such as from Quantum Devices, Inc., of Barneveld, Wis. Culture plates were placed directly on the diode array unit. Treatment comprised irradiation at 670 nm for 5 minutes resulting in a power intensity of 50 mW/cm² and an energy density of 8 joules/cm². The near-IR LED treatment was administered at 1, 15, 26 hours after MPP+ exposure. Exposure to MPP+ produced a concentration-dependent decrease in cell proliferation, mitochondrial function, and cell viability accompanied by a concentration dependent increase in reactive oxygen species production.

Three 670 nm LED (8 J/cm²) treatments significantly attenuated the cytotoxic actions of MPP+ resulting in increased cellular proliferation, profoundly enhanced mitochondrial function, reduced oxidative stress and increased viability.

Additionally, the 670 nm photon irradiation ameliorated the toxicity of the Parkinsonian drug 1-methyl-4-phenyl-1,2,3,6-tetrahydropyridine (MPTP). Mammals treated with MPTP develop the cardinal features of Parkinson's disease, akinesia and loss of dopamine in the basal ganglia, within hours. The rapid onset of the Parkinsonian syndrome following acute MPTP intoxication thus provided an excellent paradigm for the initial assessment of the therapeutic potential of near-IR photon therapy. Administering MPTP to simulate Parkinson's disease has the added advantage that it poisons the very process thought to account for the beneficial actions of near-IR light, namely mitochondrial energy production. To investigate the ability of near infrared light at 670 nm to ameliorate the acute toxicity of the Parkinsonian drug MPTP, C57BL/6 mice (20-25 g) were either pretreated with 670 nm photon irradiation or were treated subsequent to MPTP treatment. The MPTP (saline control) was administered subcutaneously at a dose of 50 mg/kg. Mice were then subjected to behavioral testing.

To administer the near-IR light, a system consistent with the above described system 10, and having LEDs arranged as will be described below was used. Specifically, the LED arrays were arranged to lie on 4 sides of a Perspex 4-mouse animal restrainer, such that the mice were illuminated from the top, bottom and both sides resulting in a power intensity of 25 mW/cm² per array and a calculated dose of 6 J/cm² per minute of exposure. For behavioral testing, plexiglass cages with white floors and translucent walls were used as the open field (26×26×39 cm). Behavioral activity was measured using infrared beams. The patterns of beam breaks were computed (Truscan Software) to obtain parameters of locomotor activity. Each animal was tested from 0-12, 23-24, 47-48, and 71-72 hours post injection.

The MPTP was metabolized to MPP+ within 5 minutes and caused a major depression of dopamine in the striatum and substantia nigra within 15 to 30 minutes. These changes suggest the major effects of MPTP are induced within the 15 to 30 minutes of its administration. A single 670 nm LED treatment of 10 minutes with a dose of 60 J/cm² was administered following MPTP, but did not alter the changes in loco-motor behavior brought about by MPTP. These studies suggested that 670 nm LED treatment was not able to reverse the effects of MPTP when given after the toxin, at least, in the paradigms tested. This finding is consistent with previous studies suggesting that the activation of cell signaling pathways, gene transcription, and protein synthesis are required for the cytoprotective actions of near-IR phototherapy.

Consequently, testing the ability of pretreatments at 670 nm to affect MPTP-injected mice showed that a pretreatment for 5 minutes (30 J/cm²) twice a day over 3 days attenuated the deficits in loco-motor behavior induced by a single injection of MPTP. The LED pretreatment attenuated the effects of MPTP on the length of time spent moving, the number of movements made, the distance moved, and the velocity. Moreover the 670 nm LED pretreatments essentially restored these behaviors to control levels by the end of a 48 hour period. Remarkably, the 670 nm LED pretreatment attenuated MPTP-induced weight loss (12% weight loss vs. 33% weight loss) and prevented the MPTP-induced death of the animals. Taken together, these observations demonstrated a clear therapeutic benefit of 670 nm LED pretreatments against the acute toxicity of MPTP.

Additionally, calculations regarding the feasibility of delivering a therapeutic dose of mid-infrared radiation to the substantia nigra were completed using a Monte Carlo simulation to approximate the diffusion of photons from a surface emitter located on the scalp through multiple layers of tissue. Optical properties at 800 nm were assumed as follows in Table 2:

| | Index of | Thickness | Reduced Scattering | Absorption |
|---|---|---|---|---|
| Layer | Refraction | [mm] | [mm−1] us * (1 − g) | [mm−1] |
| Scalp | 1.4 | 5 | 1.9 | 0.018 |
| Skull | 1.42 | 9 | 1.6 | 0.016 |
| CSF | 1.34 | 2.5 | 0.25 | 0.004 |
| Gray Matter | 1.4 | 16 | 2.2 | 0.036 |
| White Matter | 1.39 | 50 | 9.1 | 0.014 |

Instead of a curved/spherical geometry, we assumed a slab with the tabulated optical properties. Superposition of the results from the representative case for any given angle of approach should be valid with the assumption that the layered structure outlined in Table 2 is valid for any direct external path through the scalp to the mid brain. In fact, expansion of the source to include an emission surface equivalent to the surface area of the scalp (like a swimmers cap) is valid as long as the bones of the base of the skull are avoided.

From the measurements provided from imaging, it is evident that the substantia nigra resides approximately 8-9 cm deep from most external approaches; however, the final layer of the model was allowed infinite depth to “catch” all photons. For ease of scaling, a source of even irradiance (flat top) and a circular geometry (1 cm diameter) was assumed. Similarly, a total power of 1 watt was assumed at the surface, resulting in a modeled irradiance of 1.27 W/cm².

The average value over all the grids from 8-9 cm depth was 1.5×10⁻⁵ W/cm³ to 2.5×10⁻⁵ W/cm³, giving a local irradiance within the tissue of approximately 1.3×10⁻⁴ W/cm² to 1.8×10⁻⁴ W/cm². So, from the model, to achieve a desired therapeutic dose of 5 mW/cm² at a depth of 8 cm, a single 1 cm diameter source would need to have an irradiance of approximately 39 times the original 1.27 W/cm² used in the model or 49.8 W/cm². The maximum permissible exposure calculated from the ANSI standard lists for skin as 0.32 W/cm². Therefore, the required source irradiance is 156 times MPE for a 1 cm diameter source.

Based on the approximation derived via this Monte Carlo model, to achieve a therapeutic dose of 5 mW/cm², the surface area of an 800 nm source at the scalp would need to be ˜160 times larger than the source used in the simulation. Such is achievable with the above-described gantry system or a localized “bonnet” configuration. However, in the bonnet configuration some active cooling may be desirable. Alternatively, it is contemplated that a more direct approach may be used by illuminating through the roof of the nasal cavity/cribriform plate. Such could be accomplished with a source irradiance in the range of 50 mW/cm², assuming a source size at the nasal mucosa of approximately 0.8 cm².

The above described systems and methods can also be used in the treatment of other pathologies, such as cancerous tumors, for example, recurrent brain tumors. Photodynamic therapy involves the selective retention of a photosensitizer that upon activation with light mediates tumor cell destruction via the production of singlet oxygen. The cytotoxic photodynamic effect on tumor cells depends on the interaction of localized photosensitizer, light, and oxygen. Experimental and clinical studies indicate selective accumulation of photosensitizing drugs in brain tumors. In clinical practice the most common photosensitizer administered for brain tumor is hematoporphyrin derivative (HPD) and Photofrin porfimer sodium. Both of these photosensitizers are an inhomogeneous mixture of molecules that have two significant absorption peaks at 390 and 630 nm.

Light penetration into brain and tumor tissue increases with longer wavelength light. Thus, because of the infiltrative nature of many brain tumors and in particular malignant gliomas, 630 nm laser light is frequently used as a light energy source. Traditional light delivery systems that target tumor tissue are invasive and rely on fiber optics that are directly inserted into the tumor or with an inflatable balloon adapter that is placed into the resection cavity. The above-described system is capable of delivering the desire light in highly controllable dosages to the desired locations non-invasively.

The above-described systems and methods are capable of treating the entire brain, lung, liver, gastrointestianal tract, arm, leg, spinal cord or the like by providing a full-circumference photon convergence array. While radiation therapists have used surrounding arrays of gamma sources for the treatment of cancer, the present invention provides the ability to deliver near-IR (and also other light ranges) photons to large volumes of tissue to provide the light needed for healing deep inside the body. By evenly placing light source arrays all around the target, it is possible to use the physics of light convergence to our advantage while compensating for the diminishing power over distance of our current light source arrays.

The above-described systems and methods have applicability for treating a wide variety of pathologies. For example, a head injury protocol can be designed that includes the even treatment of the entire brain to minimize the effects of closed head trauma. Additionally, with respect to mucositis study, the above-described system could be used to treat the entire gastrointestianal tract, the entire spinal cord. These treatments for preventing mucositis in bone marrow transplant patients may be much more effective than traditional treatment methods because the above-described systems and methods allow treatment of the entire gastrointestinal tract. In this regard, the future of treating major musculoskeletal regions deep inside muscle and bone in arms and legs will also depend upon deep, even light delivery.

The present invention has been described in terms of the preferred embodiment, and it should be appreciated that many equivalents, alternatives, variations, and modifications, aside from those expressly stated, are possible and within the scope of the invention. Therefore, the invention should not be limited to a particular described embodiment.

1. A method for determining a minimum effective dose of irradiating light for a therapeutic process comprising the steps of:
identifying an internal target area of a patient affected by a pathology;
irradiating an externally accessible area of the patient proximate to the internal target area with a number of photons at least having wavelengths approximately within a near-infrared (IR) band;
receiving feedback from one of a spectrophotometer and a patient physiology monitoring system;
adjusting the number of photons irradiating the externally accessible area of the patient;
determining, from the feedback, the number of photons irradiating the externally accessible area of the patient that begins to cause a change in biochemical state of cytochrome oxidase in the internal target area to a desired biochemical state of cytochrome oxidase in the internal target area.

2. The method of claim 1 wherein the step of receiving feedback from the patient physiology monitoring system includes receiving one of a blood-pressure feedback and a exhalation composition feedback.

3. The method of claim 2 wherein the step of determining includes identifying a variation in blood-pressure indicative of the desired biochemical state of cytochrome oxidase in the internal target area.

4. The method of claim 2 wherein the step of determining includes identifying a concentration of nitric oxide in the exhalation composition feedback indicative of the desired biochemical state of cytochrome oxidase in the internal target area.

5. The method of claim 1 wherein the step of adjusting including incrementally increasing the number of photons irradiating the externally accessible area of the patient and the step of determining includes identifying a minimum number of photons needed to cause the change in biochemical state of cytochrome oxidase in the internal target area to the desired biochemical state of cytochrome oxidase in the internal target area.

6. The method of claim 1 wherein the step of irradiating includes arranging a plurality of light sources about the externally accessible area of the patient to direct the photons emitted by the plurality of light sources in a converging pattern toward the internal target area of the patient.

7. The method of claim 1 wherein the step of adjusting includes incrementing the number of photons to deliver a dose of energy of between 2 mW/cm² and 10 mW/cm² to the internal target area.

8. The method of claim 1 wherein the pathology includes one of Parkinson's disease and Alzheimer's disease.

9. The method of claim 1 wherein the step of irradiating the externally accessible area of the patient about the internal target area includes arranging at least one light-emitting diode (LED) within a cavity of the patient proximate to the internal target area to irradiate the externally accessible area of the patient within the cavity.

10. The method of claim 1 wherein the wavelengths of the photons are between approximately 630 nm and 670 nm.

11. A method of providing therapy for a neurodegenerative disorder comprising the steps of:
identifying an internal target area of a patient associated with the neurodegenerative disorder;
arranging a plurality of light sources about an externally accessible area of the patient proximate to an internal target area to direct photons emitted by the plurality of light sources in a converging pattern toward the internal target area of the patient;
irradiating the externally accessible area of the patient with a number of photons to deliver a dose of energy of between 2 mW/cm² and 10 mW/cm to the internal target area.

12. The method of claim 11 further comprising the step of monitoring the patient to determine the number of photons irradiating the externally accessible area of the patient that begins to cause a change in biochemical state of cytochrome oxidase in the internal target area to a desired biochemical state of cytochrome oxidase in the internal target area.

13. The method of claim 12 wherein the step of monitoring includes the step of receiving feedback from one of a spectrophotometer and a patient physiology monitoring system.

14. The method of claim 13 wherein the step of receiving feedback from the patient physiology monitoring system includes receiving one of a blood-pressure feedback and a exhalation composition feedback.

15. The method of claim 12 wherein the step of monitoring includes monitoring one of a blood-pressure of the patient and a composition of exhaled air from the patient to determine a minimum number of photons irradiating the externally accessible area of the patient that causes a change in biochemical state of cytochrome oxidase in the internal target area to a desired biochemical state of cytochrome oxidase in the internal target area.

16. A system for providing therapeutic doses of light to a target area of a patient comprising:
a plurality of light sources configured to emit photons at least having wavelengths approximately within a near-IR band configured to direct the photons emitted by the plurality of light sources in toward an internal target area of a patient during a therapy session; and
a control system configured to determine a change in biochemical state of cytochrome oxidase in the internal target area to a desired biochemical state of cytochrome oxidase in the internal target area.

17. The system of claim 16 further comprising:
a monitoring system including one of a spectrophotometer and a patient physiology monitoring system;
wherein the patient physiology monitoring system is configured to monitor one of a blood-pressure of the patient and a composition of exhaled air by the patient.

18. The system of claim 17 wherein the control system is configured to receive feedback from the monitoring system and from the feedback, determine a minimum number of photons irradiating an externally accessible area of the patient that begins to cause a change in biochemical state of cytochrome oxidase in the internal target area to a desired biochemical state of cytochrome oxidase in the internal target area.

19. A system for providing therapeutic doses of light to a target area of a patient comprising:
a plurality of light sources configured to emit photons at least having wavelengths approximately within a near-IR band configured to direct the photons emitted by the plurality of light sources in toward an internal target area of a patient during a therapy session; and
a control system configured to adjust a wavelength of the photons to cause a change in biochemical state of cytochrome oxidase in the internal target area to a desired biochemical state of cytochrome oxidase in the internal target area.

20. The system of claim 19 further comprising:
a monitoring system including one of a spectrophotometer and a patient physiology monitoring system;
wherein the patient physiology monitoring system is configured to monitor one of a blood-pressure of the patient and a composition of exhaled air by the patient;
wherein the control system is configured to receive feedback from the monitoring system and, from the feedback, determine a minimum number of photons irradiating an externally accessible area of the patient that begins to cause a change in biochemical state of cytochrome oxidase in the internal target area to a desired biochemical state of cytochrome oxidase in the internal target area.
